(19) United States
(12) Patent Application Publication
(10) Pub. No.: US 2023/0404614 A1
(43) Pub. Date: Dec. 21, 2023

(54) Orbital Atherectomy System Having An Abrasive Element

(71) Applicant: Bard Peripheral Vascular, Inc., Franklin Lakes, NJ (US)

(72) Inventors: Ikshwaku Atodaria, Phoenix, AZ (US); Andrzej J. Chanduszko, Chandler, AZ (US); Kyra Temple, Mesa, AZ (US); Garrett Repp, Gilbert, AZ (US); Faith Chamas, Scottsdale, AZ (US)

(73) Assignee: Bard Peripheral Vascular, Inc., Franklin Lakes, NJ (US)

(21) Appl. No.: 18/247,699

(22) PCT Filed: Nov. 2, 2020
(86) PCT No.: PCT/US20/58477

§ 371 Date: Apr. 3, 2023

(51) Int. Cl. A61B 17/3207

(52) U.S. Cl. CPC A61B 17/320758; A61B 2017/320004

(57) ABSTRACT

An orbital atherectomy system includes a handheld driver having a motor. An elongate flexible driveshaft has a proximal end portion and a distal end portion. The proximal end portion is drivably coupled to the motor. The distal end portion defines a rotational axis. An abrasive element is fixedly disposed on the distal end portion of the elongate flexible driveshaft. The abrasive element includes a first flat side, a second flat side, a leading tapered end portion having a leading end, and a trailing tapered end portion having a trailing end. The first flat side and the second flat side are on opposite sides of the rotational axis.

### CROSS-REFERENCE TO RELATED APPLICATIONS

None.

### TECHNICAL FIELD

The present invention relates to an atherectomy system, and, more particularly, to an orbital atherectomy system having an abrasive element.

### BACKGROUND ART

Coronary heart disease may be caused, for example, by atherosclerosis. Atherosclerosis occurs when fat, cholesterol, and/or other substances build up in the walls of blood vessels, forming hard structures called occlusions, e.g., plaques and/or atherosclerotic (stenotic) lesions. Over time, these occlusions may increase in size such that the blood vessels are substantially clogged and/or completely blocked, so as to form a total chronic occlusion (CTO).

Rotational atherectomy is a technique used to abrade, for example, calcified arterial lesions. Rotational atherectomy devices and rotational atherectomy procedures may also be referred to as rotational angioplasty devices and/or rotational angioplasty procedures. One type of rotational atherectomy device is known as an orbital atherectomy device, such as the Diamondback 360® Peripheral Orbital Atherectomy Device available from Cardiovascular Systems Inc. (“CSI”).

Rotational atherectomy devices may include an abrasive element attached to a rotatable elongate flexible driveshaft. The abrasive element may be referred to as a burr, crown, and/or bead. The rotatable elongate flexible driveshaft may be delivered over a guidewire and/or through a sheath to a desired location. The driveshaft may be rotated at high speeds (e.g., between 20,000-160,000 rpm). As the abrasive element rotates, it may be advanced over a stenotic lesion such that the abrasive element contacts the occluding plaque. In this way, the abrasive element engages the diseased lesion surface and abrades the plaque into very small particles. These small particles may be absorbed by the body or captured via the use of an embolic protection device.

What is needed in the art is an atherectomy system having an abrasive element that is better able to engage an occlusion having a small initial aperture, such as for example, an occlusion having an initial opening through which only a guidewire may pass.

### SUMMARY OF INVENTION

The present invention provides an atherectomy system having an abrasive element that is able to engage an occlusion having a small initial aperture, such as for example, an occlusion having an initial opening through which only a guidewire may pass.

The invention, in one form, is directed to an orbital atherectomy system that includes a handheld driver having a motor. An elongate flexible driveshaft has a proximal end portion and a distal end portion. The proximal end portion is drivably coupled to the motor. The distal end portion defines a rotational axis. An abrasive element is fixedly disposed on the distal end portion of the elongate flexible driveshaft. The abrasive element includes a first flat side, a second flat side, a leading tapered end portion having a leading end, and a trailing tapered end portion having a trailing end. The first flat side and the second flat side are on opposite sides of the rotational axis.

The invention, in another form, is directed to an orbital device that includes an elongate flexible driveshaft and an abrasive element. The elongate flexible driveshaft has a proximal end portion and a distal end portion. The proximal end portion is configured to be drivably coupled to a motor. The distal end portion defines a rotational axis. The abrasive element is fixedly disposed on the distal end portion of the elongate flexible driveshaft. The abrasive element includes a first flat side, a second flat side, a leading tapered end portion having a leading end, and a trailing tapered end portion having a trailing end. The first flat side and the second flat side are on opposite sides of the rotational axis.

The invention, in another form, is directed to an abrasive element for use in an orbital atherectomy system. The abrasive element, e.g., a body, includes a rotational axis, a first flat side, a second flat side, a leading tapered end portion having a leading end, and a trailing tapered end portion having a trailing end, wherein the first flat side and the second flat side are on opposite sides of the rotational axis.

An advantage of the present invention is that the flat sides of the abrasive element provide a design that is lower in mass and has less frontal surface area contact during abrasion, while maintaining the same overall cross section diameter. The lower mass and less contact area of the abrasive element helps to reduce the amount of power needed by the motor to get the system to full speed.

Another advantage of the present invention is that when the abrasive element is spun at high speeds, the abrasive element undergoes an orbital motion pathway, rather than concentric motion. In at least some embodiments, the orbital motion is characteristic of a cantilever, and is accentuated by a non-uniform mass distribution of the flat-sided abrasive element.

Another advantage is that, due to the orbital motion, the orbital device (i.e., the elongate flexible driveshaft and the abrasive element) generates a lumen of diameter in an occlusion that is larger than the outer diameter of the abrasive element.

Another advantage is that the orbital device of the invention gives the user a smooth response during activation and advancement through the occlusion.

Another advantage of the abrasive element embodiments provided by the invention is that the abrasive element may have an intermediate section interposed between two tapered portions, wherein a length of the intermediate section may be varied or eliminated as desired for optimizing weight and balance of the orbital device.

In embodiments wherein the abrasive element is mounted on the distal end of the elongate flexible driveshaft, another advantage over existing orbital atherectomy devices is that this design is better able to engage occlusions with small initial lumens or total occlusions, only needing a guidewire to have partially passed through the occlusion.

### BRIEF DESCRIPTION OF DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a schematic perspective view of an embodiment of an orbital atherectomy system having a handheld driver coupled to an orbital device having an elongate flexible driveshaft and an abrasive element, which is configured in accordance with an aspect of the present invention.

FIG. 2 is an enlarged perspective view of a portion of the orbital device of FIG. 1 taken at circle 2-2 of FIG. 1, which shows the abrasive element coupled to a distal portion of the elongate flexible driveshaft in accordance with an embodiment of the invention, wherein the abrasive element is proximal to the distal end of the elongate flexible driveshaft.

FIG. 3 is a side view of the abrasive element of FIGS. 1 and 2.

FIG. 4 is an end view of the abrasive element of FIGS. 1-3.

FIG. 5 is an enlarged side view that shows another embodiment of an orbital device suitable for use with the handheld driver of FIG. 1, wherein the distal end of the elongate flexible driveshaft terminates at or proximal to a leading end of the abrasive element.

FIG. 6 is an end view of the orbital device of FIG. 5.

FIG. 7 is a side view of another embodiment of an abrasive element that may be substituted for the abrasive element in any of the embodiments of FIGS. 1-6.

FIG. 8 is an end view of the abrasive element of FIG. 7.

FIG. 9 is a variation of the embodiment of the orbital device of FIG. 5, wherein the distal end of the abrasive element has a rounded leading end.

FIG. 10 is an end view of the orbital device of FIG. 9.

FIG. 11 is a variation of the embodiment of the orbital device of FIGS. 9 and 10, wherein the abrasive element is longitudinally asymmetrical.

FIG. 12 is an end view of the orbital device of FIG. 11.

Corresponding reference characters indicate corresponding parts throughout the several views. The exemplifications set out herein illustrate embodiments of the invention, and such exemplifications are not to be construed as limiting the scope of the invention in any manner.

### DESCRIPTION OF EMBODIMENTS

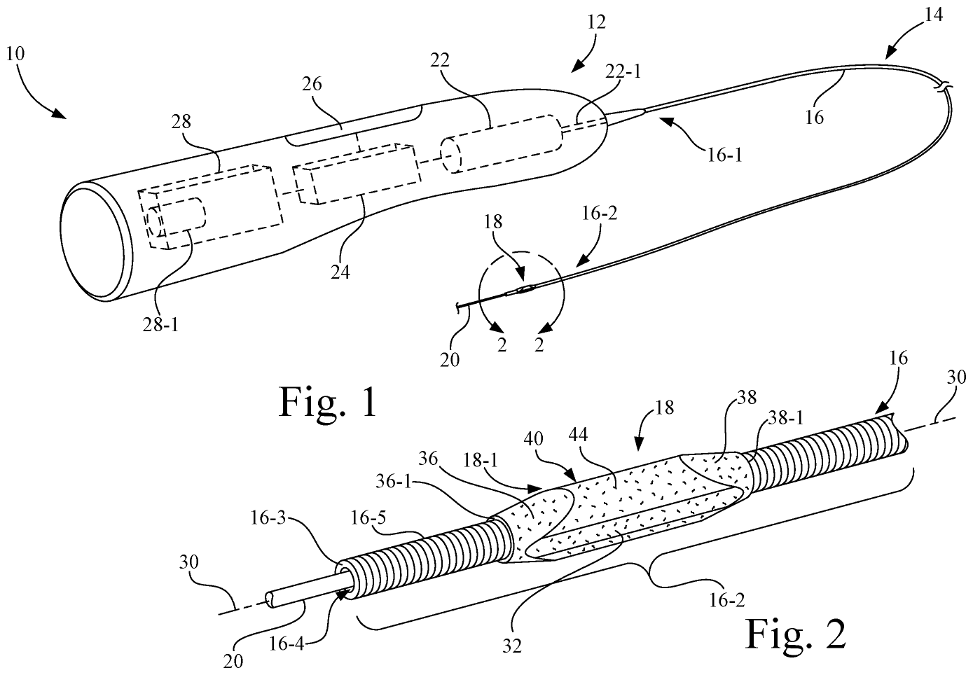

Referring now to the drawings, and more particularly to FIGS. 1 and 2, there is shown an orbital atherectomy system 10 in accordance with an embodiment of the present invention.

Orbital atherectomy system 10 includes a handheld driver 12 that is configured to rotate an orbital device 14 at high speeds (e.g., 20,000-160,000 rpm). Orbital device 14 includes an elongate flexible driveshaft 16 and an abrasive element 18. Elongate flexible driveshaft 16 if flexible so that it may be adaptable to the curvature of the vasculature. Abrasive element 18 is disposed on and fixedly (i.e., non-detachably or non-removably) attached to elongate flexible driveshaft 16. In the present embodiment, orbital device 14 may axially traverse, and rotate around, a guidewire 20. Those skilled in the art will recognize that lubricant or saline may be supplied through the elongate flexible driveshaft 16 to cool/lubricate the coil shaft/guidewire interface, as is known in the art. Abrasive element 18 performs abrasion (e.g., sanding) of an occlusion, e.g., calcified plaques and/or atherosclerotic (stenotic) lesions in the blood vessel, by high speed rotation of orbital device 14 with abrasive element 18 in contact with the occlusion, so as to reduce at least a portion of the occlusion into small particles. When abrasive element 18 is spun at high speeds, abrasive element 18 undergoes an orbital motion pathway, rather than concentric motion.

Handheld driver 12 may include a motor 22, such as a direct current (DC) motor, a motor controller circuit 24, and a user interface 26. An on-board battery power supply 28 is connected in electrical communication with motor 22, motor controller circuit 24, and user interface 26. Electrical power may be supplied to motor 22, motor controller circuit 24, and user interface 26 via on-board battery power supply 28. In the present embodiment, on-board battery power supply 28 includes a rechargeable or replaceable battery 28-1. Alternatively, an off-board power source, such as an alternating current (AC) wall outlet, may supply electrical power to handheld driver 12.

User interface 26 may be, for example, a touch screen or panel having physical or virtual buttons for supplying user input commands to motor controller circuit 24. Motor controller circuit 24 includes processing circuitry and power circuitry, so as to receive the user input commands, execute program instructions, and supply power and operational signals to motor 22. Such user input commands may include, for example, selectable rotational speed commands, motor acceleration and/or torque profile commands, and/or rotational direction commands.

Orbital device 14, including elongate flexible driveshaft 16 and abrasive element 18, is configured to extend into a blood vessel, e.g., an artery, of a patient. In the present embodiment, elongate flexible driveshaft 16 may be, for example, an elongate tightly wound metallic coil. Alternatively, elongate flexible driveshaft 16 may be a flexible metal or polymer tube. Elongate flexible driveshaft 16 includes a proximal end portion 16-1, a distal end portion 16-2, a distal end 16-3, and an elongate lumen 16-4, e.g., an elongate guidewire lumen. Distal end 16-3 is the distal terminus of distal end portion 16-2 of elongate flexible driveshaft 16.

A proximal end portion 16-1 of elongate flexible driveshaft 16 is drivably coupled, i.e., connected, e.g., directly or indirectly through a gear train, to a rotatable motor shaft 22-1 of motor 22. In the present embodiment, distal end portion 16-2 may constitute, for example, 0.5 to 10 percent of the total length of elongate flexible driveshaft 16. Distal end portion 16-2 of elongate flexible driveshaft 16 defines a rotational axis 30 about which abrasive element 18 is rotated in unison with elongate flexible driveshaft 16.

Elongate lumen 16-4 is configured, e.g., in size and shape, to accommodate guidewire 20. Elongate lumen 16-4 is configured to slidably receive guidewire 20, such that elongate flexible driveshaft 16 that carries abrasive element 18 may be axially advanced over, and rotated around, guidewire 20 and rotational axis 30. In the present embodiment, distal end portion 16-2 of elongate flexible driveshaft 16 and abrasive element 18 may be longitudinally advanceable over guidewire 20 and into the blood vessel of the patient so as to engage an occlusion in the blood vessel. Alternatively, in some embodiments, orbital atherectomy system 10 may be used in the absence of guidewire 20, wherein for example, a sheath (not shown) may be used to introduce orbital device 14 into the blood vessel of the patient.

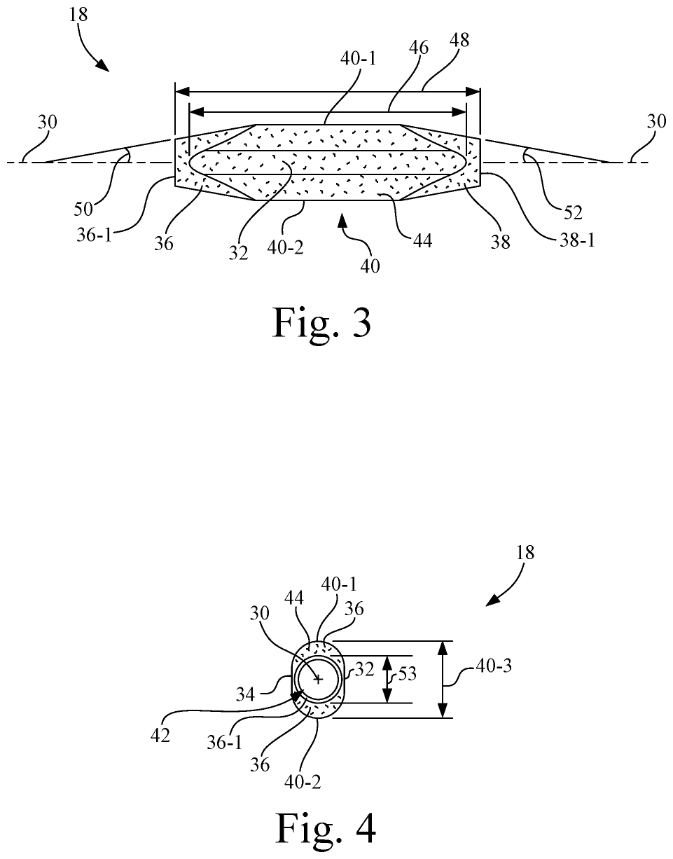

Referring also to FIGS. 3 and 4, there is shown side and end views of abrasive element 18, in isolation. Abrasive element 18 of orbital device 14 is fixedly disposed on (i.e., disposed on and fixedly attached to) distal end portion 16-2 of elongate flexible driveshaft 16. Such fixed attachment of abrasive element 18 to distal end portion 16-2 of elongate flexible driveshaft 16 may be achieved, for example, by a weld or by an adhesive.

In the embodiment of FIGS. 1 and 2, abrasive element 18 is coupled to a location in distal end portion 16-2 of elongate flexible driveshaft 16 that is proximal to distal end 16-3 of elongate flexible driveshaft 16. In the embodiment of FIGS. 1 and 2, abrasive element 18 may be coupled to elongate flexible driveshaft 16 at a location that is, for example, in a range of 8 millimeters (mm) to 25 mm proximal to distal end 16-3 of elongate flexible driveshaft 16.

In each of the embodiments depicted in FIGS. 1-6, abrasive element 18 is configured to be symmetrical around rotational axis 30, is configured to have a non-uniform mass distribution around rotational axis 30, and/or is configured to have a center of mass that lies on rotational axis 30. In accordance with an aspect of the present invention, in the present embodiment abrasive element 18 includes a first flat side 32, a second flat side 34, a leading tapered end portion 36 having a leading end 36-1, a trailing tapered end portion 38 having a trailing end 38-1, an intermediate section 40 interposed between leading tapered end portion 36 and trailing tapered end portion 38, and an elongate opening 42. As used herein, the term “flat side” means a planar surface that may include surface irregularities such as marked lumps, indentations, or elevated points, or alternatively may be smooth.

Elongate opening 42 of abrasive element 18 is configured, e.g., in size and shape, to receive, e.g., at least a portion, of elongate flexible driveshaft 16. Abrasive element 18 is fixedly attached at elongate opening 42 to distal end portion 16-2 of elongate flexible driveshaft 16, for example, by a weld or by an adhesive.

Abrasive element 18 has an exterior surface 18-1 that encompasses first flat side 32, second flat side 34, leading tapered end portion 36, trailing tapered end portion 38, and intermediate section 40, wherein at least a portion of exterior surface 18-1 is roughened, e.g., includes abrasive particles 44 (represented by stipple in the drawings). In the present embodiment, each of first flat side 32, second flat side 34, leading tapered end portion 36, trailing tapered end portion 38, and intermediate section 40 may include abrasive particles 44. However, in some applications, it may be desirable that some portions of exterior surface 18-1, e.g., first flat side 32 and second flat side 34, may be void of abrasive particles 44. As a further alternative, in some applications, it may be desirable that first flat side 32 and second flat side 34 have reduced amounts or increased amounts of abrasive particles 44 in comparison with the rest of abrasive element 18.

Abrasive particles 44 may be, for example, diamond particles present in an adhesive abrasive coating that is applied to form exterior surface 18-1 over a body substrate (e.g., a metal or polymer body) that is defined by first flat side 32, second flat side 34, leading tapered end portion 36, trailing tapered end portion 38, and intermediate section 40. Alternatively, abrasive element 18 may be formed of a compressed or bonded material having abrasive particles 44 exposed at exterior surface 18-1.

First flat side 32 and second flat side 34 are on opposite sides of rotational axis and are oppositely facing, i.e., first flat side 32 and second flat side 34 face in opposite directions. Intermediate section 40 has diametrically opposed convex side surfaces 40-1, at diameter 40-3 that are circumferentially interposed between the oppositely facing first flat side 32 and second flat side 34.

First flat side 32 and second flat side 34 may be substantially parallel, and in the present embodiment, first flat side 32 and second flat side 34 are parallel. As used herein, the term “substantially parallel” means a range that includes parallel and a permissible variation from parallel of up to plus or minus three degrees. Referring particularly to FIG. 3 with reference to FIG. 4, a longitudinal length 46 of each of first flat side 32 and second flat side 34 is less than an overall longitudinal length 48 of abrasive element 18. Overall longitudinal length 48 of abrasive element 18 may be, for example, between about 1 and mm in length. In one embodiment, for example, overall longitudinal length 48 of abrasive element 18 may be 8 mm.

In the present embodiment, leading tapered end portion 36 and trailing tapered end portion 38 of abrasive element 18 taper in opposite directions along rotational axis 30. Also, leading tapered end portion 36 and trailing tapered end portion 38 of abrasive element 18 may be axially (longitudinally) symmetrical, e.g., symmetrical with respect to a plane normal to rotational axis 30. In the present embodiment, each of leading tapered end portion 36 of abrasive element 18 and trailing tapered end portion 38 of abrasive element 18 is at least partially defined by a cone-shaped surface, wherein each cone-shaped surface adjoins and transitions to first flat side 32 and second flat side 34. The front-facing leading tapered end portion 36 allows for entry into a reduced diameter opening in the occlusion (e.g., stenotic lesion or plaque) of the blood vessel, e.g., wherein the opening essentially only needs to be large enough to accommodate the diameter of guidewire 20. The rear-facing trailing tapered end portion 38 of abrasive element 18 allows for application of lower withdrawal forces during retraction of orbital device 14 during a procedure.

Referring to FIG. 3, an angle of taper 50 of leading tapered end portion 36 of abrasive element 18 relative to rotational axis 30 may be, for example, in a range of 10 degrees to 75 degrees, and an angle of taper 52 of trailing tapered end portion 38 of abrasive element 18 relative to rotational axis 30 may be, for example, in a range of minus degrees to minus 75 degrees. As such, with reference to FIG. 4, a diameter 53 of each of leading tapered end portion 36 at leading end 36-1 and trailing tapered end portion 38 at trailing end 38-1 is less than the diameter 40-3 of intermediate section 40.

In the embodiment depicted in FIGS. 1 and 2, with reference also to FIGS. 3 and 4, leading tapered end portion 36 of abrasive element 18 terminates at a location proximal to distal end 16-3 of elongate flexible driveshaft 16. Stated differently, a sub-portion 16-5 of distal end portion 16-2 of elongate flexible driveshaft 16 distally extends from leading end 36-1 of leading tapered end portion 36 of abrasive element 18. When abrasive element 18 is spun at high speeds, abrasive element 18 undergoes an orbital motion pathway, rather than concentric motion, which is accentuated by the non-uniform mass distribution of abrasive element 18 resulting from having first flat side 32 and second flat side 34. The orbital motion of abrasive element 18 generates an opening in the occlusion of the blood vessel that is larger than a largest diameter, e.g., diameter 40-3, of abrasive element 18 itself.

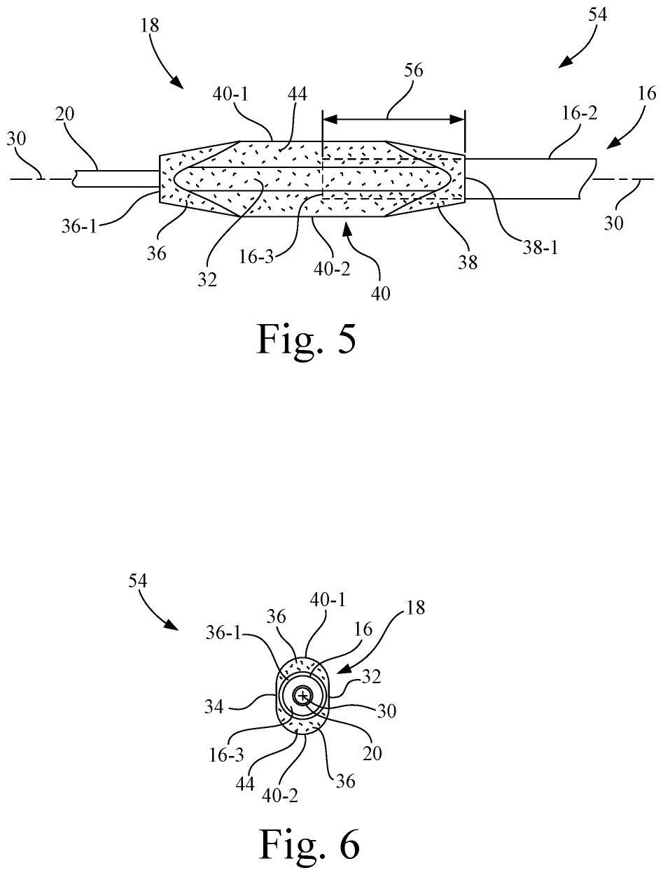

FIGS. 5 and 6 depict another embodiment utilizing elongate flexible driveshaft 16 and abrasive element 18, which together form an alternative orbital device 54. Orbital device 54 may be substituted for orbital device 14 of FIGS. 1 and 2.

In orbital device 54 depicted in FIGS. 5 and 6, distal end 16-3 of elongate flexible driveshaft 16 terminates at or proximal to leading end 36-1 of leading tapered end portion 36 of abrasive element 18. When abrasive element 18 is spun, abrasive element 18 undergoes an orbital motion pathway, rather than concentric motion. The orbital motion of abrasive element 18 on elongate flexible driveshaft 16, configured as orbital device 54, is characteristic of a cantilever, which is accentuated by the non-uniform mass distribution of abrasive element 18 resulting from first flat side 32 and second flat side 34.

A depth of insertion 56 of elongate flexible driveshaft 16 into abrasive element 18, as measured from trailing end 38-1, may be varied during assembly so as to manipulate a magnitude of the orbital motion of abrasive element 18, wherein the smaller the depth of insertion 56, the greater the magnitude of the orbital motion. The orbital motion of abrasive element 18 generates an opening in the occlusion that is larger than a largest diameter, e.g., diameter 40-3, of abrasive element 18 itself.

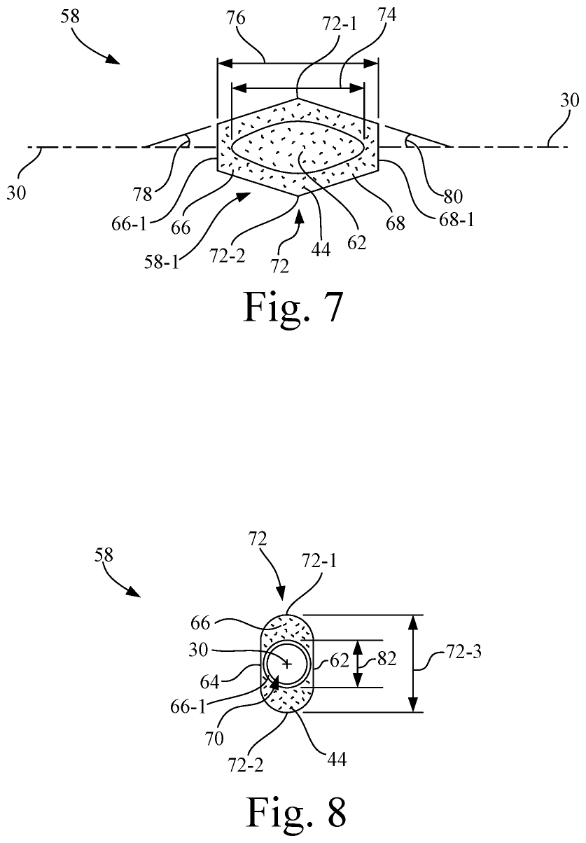

FIGS. 7 and 8 show another embodiment of a configuration for an abrasive element 58, which may be substituted for abrasive element 18 in any of the previously described embodiments associated with FIGS. 1-6. A primary difference in the overall construction of abrasive element 58 and that of abrasive element 18 is the elimination of the intermediate section 40 of abrasive element 18 (compare, for example, FIGS. 3 and 5). In the embodiment depicted in FIGS. 7 and 8, abrasive element 58 is configured to be symmetrical around rotational axis 30, is configured to have a non-uniform mass distribution around rotational axis 30, and/or is configured to have a center of mass that lies on rotational axis 30.

In the embodiment of FIGS. 7 and 8, abrasive element 58 includes a first flat side 62, a second flat side 64, a leading tapered end portion 66 having a leading end 66-1, a trailing tapered end portion 68 having a trailing end 68-1, and an elongate opening 70. Leading tapered end portion 66 of abrasive element 58 directly transitions into trailing tapered end portion 68 of abrasive element 58 to form a crowned apexes 72 having diametrically opposed curvatures 72-1, 72-2 at diameter 72-3, e.g., at a longitudinal midpoint of abrasive element 58.

Elongate opening 70 of abrasive element 58 is configured, e.g., in size and shape, to receive, e.g., at least a portion, of elongate flexible driveshaft 16. Abrasive element 58 may be fixedly attached at elongate opening 70 to distal end portion 16-2 of elongate flexible driveshaft 16, for example, by a weld or by an adhesive.

Abrasive element 58 has an exterior surface 58-1 that encompasses first flat side 62, second flat side 64, leading tapered end portion 66, and trailing tapered end portion 68, wherein at least a portion of exterior surface 58-1 is roughened, e.g., includes abrasive particles 44 (represented by stipple in the drawings). In the present embodiment, each of first flat side 62, second flat side 64, leading tapered end portion 66, and trailing tapered end portion 68 may include abrasive particles 44. However, in some applications, it may be desirable that some portions of exterior surface 58-1, e.g., first flat side 62 and second flat side 64, may be void of abrasive particles 44. As a further alternative, in some applications, it may be desirable that first flat side 62 and second flat side 64 have reduced amounts or increased amounts of abrasive particles 44 in comparison with the rest of abrasive element 18.

Abrasive particles 44 may be, for example, diamond particles present in an adhesive abrasive coating that is applied to form exterior surface 58-1 over a body substrate (e.g., a metal or polymer body) that is defined by first flat side 62, second flat side 64, leading tapered end portion 66, and trailing tapered end portion 68. Alternatively, abrasive element 58 may be formed of a compressed material having abrasive particles 44 exposed at exterior surface 58-1.

First flat side 62 and second flat side 64 are on opposite sides of rotational axis and are oppositely facing, i.e., first flat side 62 and second flat side 64 face in opposite directions. Diametrically opposed curvatures 72-1, 72-2 of crowned apexes 72 are circumferentially interposed between the oppositely facing first flat side 62 and second flat side 64.

First flat side 62 and second flat side 64 may be substantially parallel, and in the present embodiment, first flat side 62 and second flat side 64 are parallel. Referring particularly to FIG. 7 with reference to FIG. 8, a longitudinal length 74 of each of first flat side 62 and second flat side 64 is less than an overall longitudinal length 76 of abrasive element 58. Overall longitudinal length 76 of abrasive element 58 may be, for example, between about 1 and 15 mm in length. In one embodiment, for example, overall longitudinal length 76 of abrasive element 58 may be 8 mm.

In the present embodiment, leading tapered end portion 66 and trailing tapered end portion 68 of abrasive element 58 taper in opposite directions along rotational axis 30. Also, leading tapered end portion 66 and trailing tapered end portion 68 of abrasive element 58 may be axially symmetrical. In the present embodiment, each of leading tapered end portion 66 of abrasive element 58 and trailing tapered end portion 68 of abrasive element 58 is at least partially defined by a cone-shaped surface, wherein each cone-shaped surface adjoins and transitions to first flat side 62 and second flat side 64. The front-facing leading tapered end portion 66 allows for entry into a reduced diameter opening in the occlusion (e.g., stenotic lesion or plaque) of the blood vessel, e.g., wherein the opening essentially only needs to be large enough to accommodate the diameter of guidewire 20 (see also FIGS. 1 and 2 for reference). The rear-facing trailing tapered end portion 68 of abrasive element 58 allows for application of lower withdrawal forces during retraction of the orbital device during a procedure.

Referring to FIG. 7, an angle of taper 78 of leading tapered end portion 66 of abrasive element 58 relative to rotational axis 30 may be, for example, in a range of 10 degrees to 75 degrees, and an angle of taper 80 of trailing tapered end portion 68 of abrasive element 58 relative to rotational axis 30 may be, for example, in a range of minus 10 degrees to minus 75 degrees. As such a diameter 82 of each of leading tapered end portion 66 at leading end 66-1 and trailing tapered end portion 68 at trailing end 68-1 is less than the diameter 72-3 of crowned apexes 72.

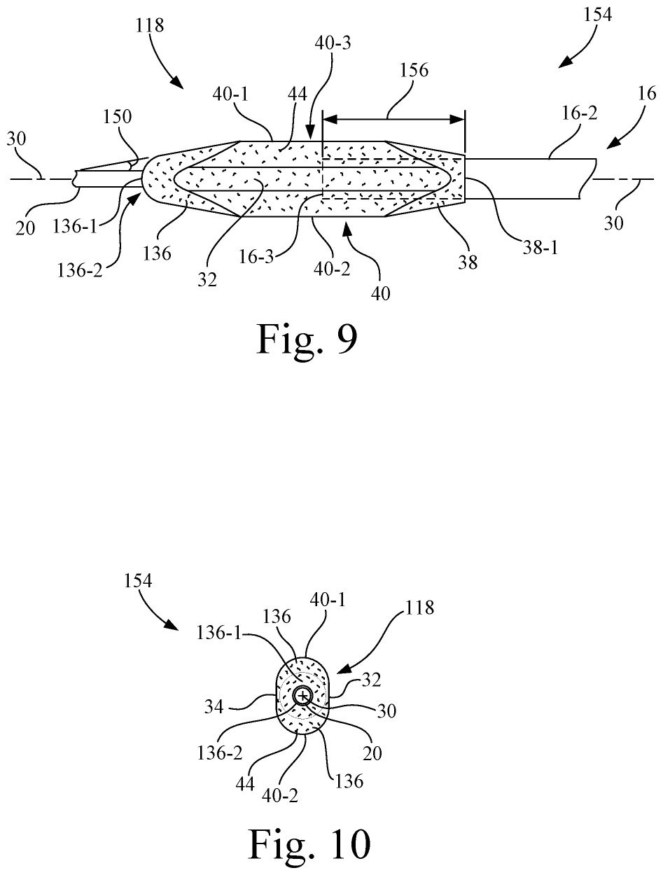

FIGS. 9 and 10 depict an embodiment utilizing elongate flexible driveshaft 16 and an abrasive element 118, which together form an alternative orbital device 154. Orbital device 154 may be substituted for orbital device 14 of FIGS. 1 and 2. Orbital device 154 is a variation of orbital device 54 of FIGS. 5 and 6.

Abrasive element 118 includes first flat side 32, second flat side 34, trailing tapered end portion 38 having trailing end 38-1, intermediate section 40, and an elongate opening 42 as in the embodiment of abrasive element 18 (see, e.g., FIGS. 1-6). However, abrasive element 118 includes a leading tapered end portion 136 having a rounded leading end 136-1 and a guidewire opening 136-2 lying on rotational axis 30. Intermediate section 40 is interposed between leading tapered end portion 136 and trailing tapered end portion 38. Guidewire opening 136-2 is sized and shaped to slidably receive guidewire 20.

In the present embodiment, leading tapered end portion 136 of abrasive element 118 is at least partially defined by a cone-shaped surface, wherein the cone-shaped surface adjoins and transitions to first flat side 32 and second flat side 34. An angle of taper 150 of leading tapered end portion 136 of abrasive element 118 relative to rotational axis 30 may be, for example, in a range of 10 degrees to 75 degrees. Rounded leading end 136-1 has a rounded, e.g., hemispherical, surface. Rounded leading end 136-1 may provide additional benefit in entering an occlusion by virtue of the rounded surface, which begins a smooth transition from guidewire 20 to the tapered surface of leading tapered end portion 136.

A depth of insertion 156 of elongate flexible driveshaft 16 into abrasive element 118, as measured from trailing end 38-1, may be varied during assembly so as to manipulate a magnitude of the orbital motion of abrasive element 118, wherein the smaller the depth of insertion 156, the greater the magnitude of the orbital motion. The orbital motion of abrasive element 118 generates an opening in the occlusion that is larger than a largest diameter, e.g., diameter 40-3, of abrasive element 118 itself.

The orbital motion of abrasive element 118 on elongate flexible driveshaft 16, configured as orbital device 154, is characteristic of a cantilever, which is accentuated by the non-uniform mass distribution of abrasive element 118 resulting from first flat side 32 and second flat side 34.

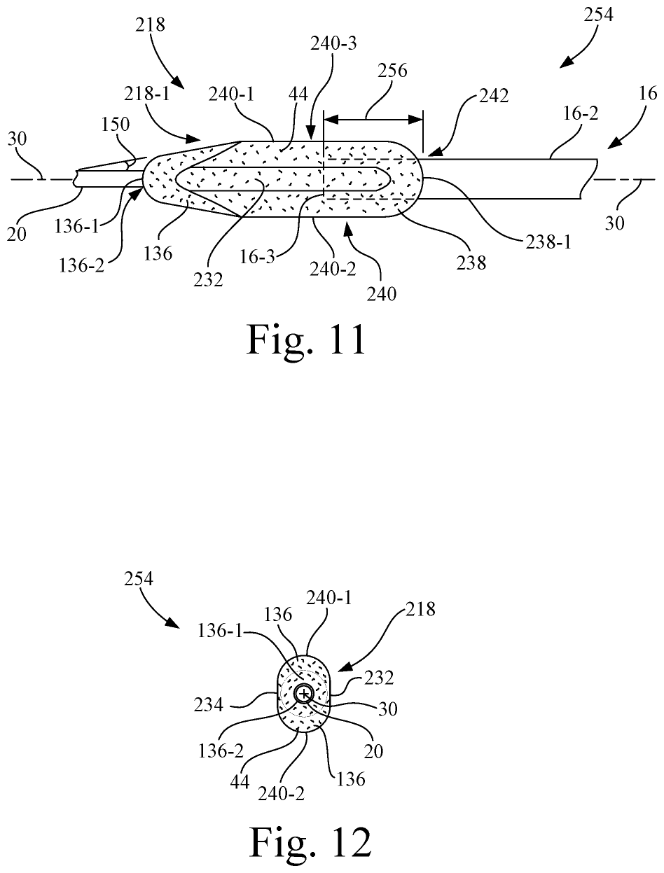

FIGS. 11 and 12 depict an embodiment utilizing elongate flexible driveshaft 16 and an abrasive element 218, which together form an alternative orbital device 254. Orbital device 254 may be substituted for orbital device 14 of FIGS. 1 and 2. Orbital device 254 is a variation of orbital device 154 of FIGS. 9 and 10.

In the present embodiment abrasive element 218 has a longitudinally asymmetrical configuration that includes a first flat side 232, a second flat side 234, leading tapered end portion 136 having leading end 136-1 (see also FIG. 9), a trailing end portion 238 having a trailing end 238-1, an intermediate section 240 interposed between leading tapered end portion 136 and trailing end portion 238, and an elongate opening 242.

Elongate opening 242 of abrasive element 218 is configured, e.g., in size and shape, to receive, e.g., at least a portion, of elongate flexible driveshaft 16. Abrasive element 218 is fixedly attached at elongate opening 242 to distal end portion 16-2 of elongate flexible driveshaft 16, for example, by a weld or by an adhesive.

Abrasive element 218 has an exterior surface 218-1 that encompasses first flat side 232, second flat side 234, leading tapered end portion 136, trailing end portion 238, and intermediate section 240, wherein at least a portion of exterior surface 218-1 is roughened, e.g., includes abrasive particles 44 (represented by stipple in the drawings). In the present embodiment, each of first flat side 232, second flat side 234, leading tapered end portion 136, trailing end portion 238, and intermediate section 240 may include abrasive particles 44. However, in some applications, it may be desirable that some portions of exterior surface 218-1, e.g., first flat side 232 and second flat side 234, may be void of abrasive particles 44. As a further alternative, in some applications, it may be desirable that first flat side 232 and second flat side 234 have reduced amounts or increased amounts of abrasive particles 44 in comparison with the rest of abrasive element 218.

Abrasive particles 44 may be, for example, diamond particles present in an adhesive abrasive coating that is applied to form exterior surface 218-1 over a body substrate (e.g., a metal or polymer body) that is defined by first flat side 232, second flat side 234, leading tapered end portion 136, trailing end portion 238, and intermediate section 240. Alternatively, abrasive element 218 may be formed of a compressed or bonded material having abrasive particles 44 exposed at exterior surface 218-1.

First flat side 232 and second flat side 234 are on opposite sides of rotational axis 30 and are oppositely facing, i.e., first flat side 232 and second flat side 234 face in opposite directions, and may be substantially identical. Intermediate section 240 has diametrically opposed convex side surfaces 240-1, 240-2 at diameter 240-3 that are circumferentially interposed between the oppositely facing first flat side 232 and second flat side 234.

First flat side 232 and second flat side 234 may be substantially parallel, and in the present embodiment, first flat side 232 and second flat side 234 are parallel. A longitudinal length of each of first flat side 232 and second flat side 234 is less than an overall longitudinal length of abrasive element 218. The overall longitudinal length of abrasive element 218 may be, for example, between about 1 and 15 mm in length. In one embodiment, for example, the overall longitudinal length of abrasive element 218 may be 8 mm.

In the present embodiment, leading tapered end portion 136 and trailing end portion 238 of abrasive element 218 face in opposite directions along rotational axis 30. Also, leading tapered end portion 136 and trailing end portion 238 of abrasive element 218 are axially (longitudinally) asymmetrical.

In the present embodiment, leading tapered end portion 136 of abrasive element 218 is at least partially defined by a cone-shaped surface, wherein the cone-shaped surface adjoins and transitions to first flat side 232 and second flat side 234. An angle of taper 150 of leading tapered end portion 136 of abrasive element 218 relative to rotational axis may be, for example, in a range of 10 degrees to 75 degrees.

Trailing end portion 238 of abrasive element 218 is a rounded, e.g., hemispherical, surface that adjoins and transitions to first flat side 232 and second flat side 234. The front-facing leading tapered end portion 136 allows for entry into a reduced diameter opening in the occlusion (e.g., stenotic lesion or plaque) of the blood vessel, e.g., wherein the opening essentially only needs to be large enough to accommodate the diameter of guidewire 20. The rear-facing trailing end portion 238 of abrasive element 218 allows for application of lower withdrawal forces during retraction of orbital device 254 during a procedure.

A depth of insertion 256 of elongate flexible driveshaft 16 into abrasive element 218, as measured from trailing end 238-1, may be varied during assembly so as to manipulate a magnitude of the orbital motion of abrasive element 218, wherein the smaller the depth of insertion 256, the greater the magnitude of the orbital motion. The orbital motion of abrasive element 218 generates an opening in the occlusion that is larger than a largest diameter, e.g., diameter 240-3, of abrasive element 218 itself.

The orbital motion of abrasive element 218 on elongate flexible driveshaft 16, configured as orbital device 254, is characteristic of a cantilever, which is accentuated by the non-uniform mass distribution of abrasive element 218 resulting from first flat side 232 and second flat side 234.

The following items also relate to the invention:

In one embodiment, the invention relates to an orbital atherectomy system. The orbital atherectomy system may include a handheld driver, an elongate (flexible) driveshaft, and an abrasive element. The handheld driver may have a motor. The elongate flexible driveshaft may have a proximal end portion and a distal end portion. The proximal end portion may be drivably coupled or is configured for drivably coupling to the motor. The distal end portion defines a rotational axis. The abrasive element may be (fixedly) disposed on the distal end portion of the elongate flexible driveshaft. The abrasive element may include a first flat side, a second flat side, a leading tapered end portion having a leading end, and a trailing (tapered) end portion having a trailing end. The first flat side and the second flat side are on opposite sides of the rotational axis/on opposite circumferential sides of the abrasive element.

In accordance with any of the embodiments, the abrasive element may have an exterior surface that encompasses the first flat side, the second flat side, the leading tapered end portion, and the trailing (tapered) end portion. At least a portion of the exterior surface includes abrasive particles.

In accordance with any of the embodiments, the first flat side and the second flat side may be (substantially) parallel.

In accordance with any of the embodiments, a longitudinal length of each of the first flat side and the second flat side may be less than an overall longitudinal length of the abrasive element.

In accordance with some embodiments, the leading tapered end portion and the trailing (tapered) end portion of the abrasive element taper in opposite directions along the rotational axis.

In accordance with embodiments having a leading tapered end portion and a trailing tapered end portion, an angle of taper of the leading tapered end portion of the abrasive element relative to the rotational axis may be in a range of 10 degrees to 75 degrees, and an angle of taper of the trailing tapered end portion of the abrasive element relative to the rotational axis may be in a range of minus 10 degrees to minus 75 degrees.

In accordance with any of the embodiments, the abrasive element may be (configured to be) symmetrical around the rotational axis, (to) have a non-uniform mass distribution around the rotational axis, and (to) have a center of mass that lies on the rotational axis.

In accordance with any of the embodiments, the distal end portion of the elongate flexible driveshaft includes a distal end of the elongate flexible driveshaft. In accordance with some embodiments, the leading tapered end portion of the abrasive element may terminate at a location proximal to the distal end of the elongate flexible driveshaft. Also, in accordance with other embodiments, a sub-portion of the distal end portion of the elongate flexible driveshaft distally may extend from the leading tapered end portion of the abrasive element.

In accordance with some embodiments, the abrasive element may include an intermediate section interposed between the leading tapered end portion and the trailing (tapered) end portion. The intermediate section may have diametrically opposed convex side surfaces that are circumferentially interposed between the oppositely facing first flat side and the second flat side.

In accordance with some embodiments, the leading tapered end portion of the abrasive element may have a leading end, the elongate flexible driveshaft may have a distal end at a distal terminus of the distal end portion of the elongate flexible driveshaft, and the distal end of the elongate flexible driveshaft may terminate at or proximal to the leading end of the leading tapered end portion of the abrasive element.

In accordance with some embodiments, the leading tapered end portion of the abrasive element may directly transition into the trailing (tapered) end portion of the abrasive element.

In accordance with embodiments having a leading tapered end portion and a trailing tapered end portion, each of the leading tapered end portion of the abrasive element and the trailing tapered end portion of the abrasive element may be at least partially defined by a cone-shaped surface. Each cone-shaped surface may adjoin and transition to the first flat side and the second flat side.

In accordance with any of the embodiments, the abrasive element may include an elongate opening configured to receive the elongate flexible driveshaft. The abrasive element may be fixedly attached at the elongate opening to the distal end portion of the elongate flexible driveshaft by a weld or by an adhesive.

In accordance with any of the embodiments, the embodiments may optionally include a guidewire, and wherein the elongate flexible driveshaft may have an elongate guidewire lumen that may be configured to slidably receive the guidewire. The elongate flexible driveshaft may be configured to be axially advanced over and rotated around the guidewire.

In another embodiment, the invention relates to an orbital device that may include an elongate flexible driveshaft and an abrasive element, and optionally the orbital atherectomy system of the preceding paragraphs. The elongate flexible driveshaft may have a proximal end portion and a distal end portion. The proximal end portion may be configured to be drivably coupled to a motor. The distal end portion may define a rotational axis. The abrasive element may be fixedly disposed on the distal end portion of the elongate flexible driveshaft. The abrasive element includes a first flat side, a second flat side, a leading tapered end portion having a leading end, and a trailing (tapered) end portion having a trailing end. The first flat side and the second flat side are on opposite sides of the rotational axis.

In accordance with any of the embodiments, the abrasive element may have an exterior surface that encompasses the first flat side, the second flat side, the leading tapered end portion, and the trailing (tapered) end portion. At least a portion of the exterior surface includes abrasive particles.

In accordance with any of the embodiments, the first flat side and the second flat side may be (substantially) parallel.

In accordance with any of the embodiments, a longitudinal length of each of the first flat side and the second flat side may be less than an overall longitudinal length of the abrasive element.

In accordance with embodiments having a leading tapered end portion and a trailing tapered end portion, the leading tapered end portion and the trailing tapered end portion of the abrasive element taper in opposite directions along the rotational axis.

In accordance with embodiments having a leading tapered end portion and a trailing tapered end portion, an angle of taper of the leading tapered end portion of the abrasive element relative to the rotational axis may be in a range of 10 degrees to 75 degrees, and an angle of taper of the trailing tapered end portion of the abrasive element relative to the rotational axis may be in a range of minus 10 degrees to minus 75 degrees.

In accordance with any of the embodiments, the abrasive element may be (configured to be) symmetrical around the rotational axis, (to) have a non-uniform mass distribution around the rotational axis, and (to) have a center of mass that lies on the rotational axis.

In accordance with any of the embodiments, the distal end portion of the elongate flexible driveshaft includes a distal end of the elongate flexible driveshaft. In accordance with some embodiments, the leading tapered end portion of the abrasive element may terminate at a location proximal to the distal end of the elongate flexible driveshaft. Also, in accordance with other embodiments, a sub-portion of the distal end portion of the elongate flexible driveshaft distally extends from the leading tapered end portion of the abrasive element.

In accordance with some embodiments, the abrasive element may include an intermediate section interposed between the leading tapered end portion and the trailing (tapered) end portion. The intermediate section may have diametrically opposed convex side surfaces that are circumferentially interposed between the oppositely facing first flat side and the second flat side.

In accordance with some embodiments, the leading tapered end portion of the abrasive element may have a leading end, the elongate flexible driveshaft may have a distal end at a distal terminus of the distal end portion of the elongate flexible driveshaft, and the distal end of the elongate flexible driveshaft may terminate at or proximal to the leading end of the leading tapered end portion of the abrasive element.

In accordance with some embodiments, the leading tapered end portion of the abrasive element directly transitions into the trailing (tapered) end portion of the abrasive element.

In accordance with embodiments having a leading tapered end portion and a trailing tapered end portion, each of the leading tapered end portion of the abrasive element and the trailing tapered end portion of the abrasive element may be at least partially defined by a cone-shaped surface. Each cone-shaped surface may adjoin and transition to the first flat side and the second flat side.

In accordance with any of the embodiments, the abrasive element may include an elongate opening configured to receive the elongate flexible driveshaft. The abrasive element may be fixedly attached at the elongate opening to the distal end portion of the elongate flexible driveshaft by a weld or by an adhesive.

In accordance with any of the embodiments, the embodiments may optionally include a guidewire, and wherein the elongate flexible driveshaft may have an elongate guidewire lumen that may be configured to slidably receive the guidewire. The elongate flexible driveshaft may be configured to be axially advanced over and rotated around the guidewire.

In another embodiment, the invention relates to an abrasive element for use in and/or configured for an orbital atherectomy system. The abrasive element may include a rotational axis, a first flat side, a second flat side, a leading end portion having a leading end, and a trailing end portion having a trailing end. The first flat side and the second flat side are on opposite sides of the rotational axis.

In accordance with some embodiments, at least one of the leading end portion and the trailing end portion may be rounded.

In accordance with some embodiments, the abrasive element may be longitudinally symmetrical.

In accordance with some embodiments, the abrasive element may be longitudinally asymmetrical.

As used herein, and unless stated otherwise or supplemental to in the context of its use, the term “substantially”, “about”, and other words of degree are relative modifiers intended to indicate permissible variation from the characteristic so modified, possessing more of the physical or functional characteristic than its opposite, and approaching or approximating such a physical or functional characteristic.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An orbital atherectomy system, comprising:
a handheld driver having a motor;
an elongate flexible driveshaft having a proximal end portion and a distal end portion, the proximal end portion being drivably coupled to the motor, the distal end portion defining a rotational axis; and
an abrasive element fixedly disposed on the distal end portion of the elongate flexible driveshaft, wherein the abrasive element includes a first flat side, a second flat side, a leading tapered end portion having a leading end, and a trailing tapered end portion having a trailing end, wherein the first flat side and the second flat side are on opposite sides of the rotational axis.

2. The orbital atherectomy system according to claim 1, wherein the abrasive element has an exterior surface that encompasses the first flat side, the second flat side, the leading tapered end portion, and the trailing tapered end portion, wherein at least a portion of the exterior surface includes abrasive particles.

3. The orbital atherectomy system of claim 1, wherein the first flat side and the second flat side are substantially parallel.

4. The orbital atherectomy system of claim 1, wherein a longitudinal length of each of the first flat side and the second flat side is less than an overall longitudinal length of the abrasive element.

5. The orbital atherectomy system of claim 1, wherein the leading tapered end portion and the trailing tapered end portion of the abrasive element taper in opposite directions along the rotational axis.

6. The orbital atherectomy system to of claim 1, wherein an angle of taper of the leading tapered end portion of the abrasive element relative to the rotational axis is in a range of 10 degrees to 75 degrees, and an angle of taper of the trailing tapered end portion of the abrasive element relative to the rotational axis is in a range of minus 10 degrees to minus 75 degrees.

7. The orbital atherectomy system according of claim 1, wherein the abrasive element is configured:
to be symmetrical around the rotational axis,
to have a non-uniform mass distribution around the rotational axis, and
to have a center of mass that lies on the rotational axis.

8. The orbital atherectomy system of claim 1, wherein the distal end portion of the elongate flexible driveshaft includes a distal end of the elongate flexible driveshaft, and wherein the leading tapered end portion of the abrasive element terminates at a location proximal to the distal end of the elongate flexible driveshaft.

9. The orbital atherectomy system of claim 1, wherein a sub-portion of the distal end portion of the elongate flexible driveshaft distally extends from the leading tapered end portion of the abrasive element.

10. The orbital atherectomy system of claim 1, wherein the abrasive element includes an intermediate section interposed between the leading tapered end portion and the trailing tapered end portion, the intermediate section having diametrically opposed convex side surfaces that are circumferentially interposed between the oppositely facing first flat side and the second flat side.

11. The orbital atherectomy system of claim 1, wherein:
the leading tapered end portion of the abrasive element has a leading end,
the elongate flexible driveshaft has a distal end at a distal terminus of the distal end portion of the elongate flexible driveshaft, and
the distal end of the elongate flexible driveshaft terminates at or proximal to the leading end of the leading tapered end portion of the abrasive element.

12. The orbital atherectomy system of claim 1, wherein the leading tapered end portion of the abrasive element directly transitions into the trailing tapered end portion of the abrasive element.

13. The orbital atherectomy system of claim 1, wherein each of the leading tapered end portion of the abrasive element and the trailing tapered end portion of the abrasive element is at least partially defined by a cone-shaped surface, wherein each cone-shaped surface adjoins and transitions to the first flat side and the second flat side.

14. The orbital atherectomy system of claim 1, wherein the abrasive element includes an elongate opening configured to receive the elongate flexible driveshaft, the abrasive element being fixedly attached at the elongate opening to the distal end portion of the elongate flexible driveshaft by a weld or by an adhesive.

15. The orbital atherectomy system of claim 1, comprising a guidewire, and wherein the elongate flexible driveshaft has an elongate guidewire lumen that is configured to slidably receive the guidewire, the elongate flexible driveshaft configured to be axially advanced over and rotated around the guidewire.

16. An orbital device, comprising:
an elongate flexible driveshaft having a proximal end portion and a distal end portion, the proximal end portion configured to be drivably coupled to a motor, the distal end portion defining a rotational axis; and
an abrasive element fixedly disposed on the distal end portion of the elongate flexible driveshaft, wherein the abrasive element includes a first flat side, a second flat side, a leading tapered end portion having a leading end, and a trailing tapered end portion having a trailing end, wherein the first flat side and the second flat side are on opposite sides of the rotational axis.

17. The orbital device according to claim 16, wherein the abrasive element has an exterior surface that encompasses the first flat side, the second flat side, the leading tapered end portion, and the trailing tapered end portion, wherein at least a portion of the exterior surface includes abrasive particles.

18. The orbital device of claim 16, wherein the first flat side and the second flat side are substantially parallel.

19. The orbital device of claim 16, wherein a longitudinal length of each of the first flat side and the second flat side is less than an overall longitudinal length of the abrasive element.

20. The orbital device of claim 16, wherein the leading tapered end portion and the trailing tapered end portion of the abrasive element taper in opposite directions along the rotational axis.

21. The orbital device of claim 16, wherein an angle of taper of the leading tapered end portion of the abrasive element relative to the rotational axis is in a range of 10 degrees to 75 degrees, and an angle of taper of the trailing tapered end portion of the abrasive element relative to the rotational axis is in a range of minus degrees to minus 75 degrees.

22. The orbital device of claim 16, wherein the abrasive element is configured:
to be symmetrical around the rotational axis,
to have a non-uniform mass distribution around the rotational axis, and
to have a center of mass that lies on the rotational axis.

23. The orbital device of claim 16, wherein the distal end portion of the elongate flexible driveshaft includes a distal end of the elongate flexible driveshaft, and wherein the leading tapered end portion of the abrasive element terminates at a location proximal to the distal end of the elongate flexible driveshaft.

24. The orbital device of claim 16, wherein a sub-portion of the distal end portion of the elongate flexible driveshaft distally extends from the leading tapered end portion of the abrasive element.

25. The orbital device of claim 16, wherein the abrasive element includes an intermediate section interposed between the leading tapered end portion and the trailing tapered end portion, the intermediate section having diametrically opposed convex side surfaces that are circumferentially interposed between the oppositely facing first flat side and the second flat side.

26. The orbital device of claim 16, wherein:
the leading tapered end portion of the abrasive element has a leading end,
the elongate flexible driveshaft has a distal end at a distal terminus of the distal end portion of the elongate flexible driveshaft, and
the distal end of the elongate flexible driveshaft terminates at or proximal to the leading end of the leading tapered end portion of the abrasive element.

27. The orbital device of claim 16, wherein the leading tapered end portion of the abrasive element directly transitions into the trailing tapered end portion of the abrasive element.

28. The orbital device of claim 16, wherein each of the leading tapered end portion of the abrasive element and the trailing tapered end portion of the abrasive element is at least partially defined by a cone-shaped surface, wherein each cone-shaped surface adjoins and transitions to the first flat side and the second flat side.

29. The orbital device of claim 16, wherein the abrasive element includes an elongate opening configured to receive the elongate flexible driveshaft, the abrasive element being fixedly attached at the elongate opening to the distal end portion of the elongate flexible driveshaft by a weld or by an adhesive.

30. The orbital device of claim 16, comprising a guidewire, and wherein the elongate flexible driveshaft has an elongate guidewire lumen that is configured to slidably receive the guidewire, the elongate flexible driveshaft configured to be axially advanced over and rotated around the guidewire.

31. An abrasive element for use in an orbital atherectomy system, comprising a rotational axis, a first flat side, a second flat side, a leading end portion having a leading end, and a trailing end portion having a trailing end, wherein the first flat side and the second flat side are on opposite sides of the rotational axis.

32. The abrasive element according to claim 31, wherein at least one of the leading end portion and the trailing end portion is rounded.

33. The abrasive element of claim 31, wherein the abrasive element is longitudinally symmetrical.

34. The abrasive element of claim 31, wherein the abrasive element is longitudinally asymmetrical.
